(19) United States
(12) Patent Application Publication
(10) Pub. No.: US 2023/0027277 A1
(43) Pub. Date: Jan. 26, 2023

(54) INTEGRATED CONVERSION AND OLIGOMERIZATION OF BIO-DERIVED ALCOHOLS

(71) Applicant: ExxonMobil Technology and Engineering Company, Annandale, NJ (US)

(72) Inventors: Matthew T. Kapelewski, Flemington, NJ (US); Lei Zhang, Basking Ridge, NJ (US); Brandon J. O'Neill, Spring, TX (US); Arsam Behkish, Flemington, NJ (US)

(21) Appl. No.: 17/852,841

(22) Filed: Jun. 29, 2022

Related U.S. Application Data
(60) Provisional application No. 63/203,323, filed on Jul. 16, 2021.

(51) Int. Cl. C07C 1/24; C12P 19/04; C12P 7/14; C07C 2/12; B01J 38/00; B01J 29/70; C12M 1/00; C10G 3/00; B01J 8/02

(52) U.S. Cl. CPC C07C 1/24; C12P 19/04; C12P 7/14; C07C 2/12; B01J 38/00; B01J 29/703; C12M 21/12; C12M 43/02; C10G 3/49; C12M 43/08; B01J 8/0278; C10G 2300/1011; C07C 2529/70; C10G 2400/04

(57) ABSTRACT

Systems and methods are provided for integrated conversion of biomass to ultimately form naphtha and/or diesel boiling range products. The integrated conversion can include an initial conversion of biomass to alcohols, such as by fermentation, followed by conversion of alcohols to olefins and then olefins to naphtha, jet, and diesel boiling range compounds, with high selectivity for formation of diesel boiling range compounds. The integrated conversion process can be facilitated by using a common catalyst for both the conversion of alcohols to olefins and the conversion of olefins to naphtha and/or diesel boiling range compounds. For example, ZSM-48 (an MRE zeotype framework structure catalyst) can be used as the catalyst for both conversion of alcohols to olefins and for oligomerization of olefins with increased selectivity for formation of diesel boiling range products.

### CROSS-REFERENCE TO RELATED APPLICATION

This application claims the priority to and the benefit of U.S. Ser. No. 63/203,323, filed Jul. 16, 2021, which is incorporated herein by reference.

### FIELD OF THE INVENTION

Systems and methods are provided for integrated conversion and subsequent oligomerization of bio-derived alcohols and/or ethers using zeolite-based catalysts.

### BACKGROUND OF THE INVENTION

Conversion of methanol to olefins and other unsaturated compounds is a commonly used reaction scheme for chemical manufacture. Conventional methods can involve exposing a methanol-containing feed to a molecular sieve, such as ZSM-5 or SAPO-34.

Alcohols, especially those produced from biological sources, are potential alternatives to petroleum-based fuels. For example, ethanol can be derived from the fermentation of biological feedstocks, as well as the bio-conversion of waste streams from steel manufacturing and of municipal solid waste. As ethanol is not a drop-in replacement for gasoline or diesel, however, it must be further upgraded to be used as a fuel in most applications. This can present several challenges. For example, because multiple conversion processes are involved in transforming a biological feedstock to an end product, yield and/or efficiency of use can be a concern. Additionally, even if non-catalyzed processes are used to convert biomass to alcohols, multiple catalytic steps can remain in order to convert the alcohol into a desired product. This can require substantial amounts of equipment to provide the reactors and supporting equipment for performing the multiple catalytic processing steps.

Narula C. K. et al (Scientific Reports volume 5, Article number: 16039, 2015) describe the conversion of ethanol to olefins over an InV-ZSM-5 zeolite. The catalyst converts ethanol at 360° C. to 6.5% olefins, 33.2% paraffins, and 60.2% aromatics.

### SUMMARY OF THE INVENTION

In an aspect, a method for converting biomass is provided. The method includes exposing a feed containing biomass to an enzymatic conversion process to form an effluent including one or more sugars. The method further includes exposing at least a portion of the one or more sugars to fermentation conditions to form a fermented effluent including one or more alcohols. The method further includes exposing a conversion stage feed comprising at least a portion of the one or more alcohols to a catalyst including ZSM-48, an MRE framework structure, or a combination thereof, under alcohol conversion conditions to form a conversion effluent including C₃₊ olefins, the alcohol conversion stage comprising a moving bed reactor, a fluidized bed reactor, a riser reactor, or a combination thereof. The method further includes exposing at least a portion of the conversion effluent to oligomerization conditions in the presence of additional catalyst including ZSM-48, an MRE framework structure, or a combination thereof, to form at least a diesel boiling range fraction, the oligomerization stage including a moving bed reactor, a fluidized bed reactor, a riser reactor, or a combination thereof. The method further includes regenerating at least a portion of the catalyst including ZSM-48, an MRE framework structure, or a combination thereof, and at least a portion of the additional catalyst including ZSM-48, an MRE framework structure, or a combination thereof, in a regenerator vessel to form regenerated catalyst. Additionally, the method includes passing at least a first portion of the regenerated catalyst into the alcohol conversion stage and at least a second portion of the regenerated catalyst into the oligomerization stage.

In another aspect, a method for converting biomass is provided. The method includes exposing a feed including biomass to an enzymatic conversion process to form an effluent including one or more sugars. The method further includes exposing at least a portion of the one or more sugars to fermentation conditions to form a fermented effluent including one or more alcohols, and a fermentation residue. The method further includes exposing a conversion stage feed including at least a portion of the one or more alcohols to a catalyst including ZSM-48, an MRE framework structure, or a combination thereof, under alcohol conversion conditions in an alcohol conversion stage to form a conversion effluent. The method further includes separating an olefin-containing fraction including C₃₊ olefins and a methane-containing fraction from the conversion effluent. The method further includes exposing at least a portion of the conversion effluent to oligomerization conditions in the presence of a catalyst including ZSM-48, an MRE framework structure, or a combination thereof, in an oligomerization stage to form at least a diesel boiling range fraction. The method further includes forming a fuel from at least a portion of the methane-containing fraction from the conversion effluent and at least a portion of the fermentation residue. Additionally, the method includes combusting at least a portion of the fuel to generate steam, to power a turbine for generating electricity, or a combination thereof.

### BRIEF DESCRIPTION OF THE DRAWINGS

FIG. 1 shows an example of an integrated system for conversion of biomass into gasoline and/or diesel boiling range products.

FIG. 2 is a graph showing total hydrocarbon (HC) yields for aromatics, paraffins, and olefins over ZSM-48 at 350° C.

FIG. 3 is a graph showing total hydrocarbon (HC) yields for aromatics, paraffins, and olefins over ZSM-48 at 450° C.

FIG. 4 is a graph showing olefin yields as a percent of the total hydrocarbon product for ZSM-48 at 350° C.

FIG. 5 is a graph showing further breakdown of olefin yields as a percent of the total hydrocarbon product for ZSM-48 at 350° C.

FIG. 6 is a graph showing olefin yields as a percent of the total hydrocarbon product for ZSM-48 at 450° C.

FIG. 7 is a graph showing total hydrocarbon (HC) yields for aromatics, paraffins, and olefins over ZSM-48 at 300° C. utilizing 40% ethanol feed.

FIG. 8 is a graph showing total hydrocarbon (HC) yields for aromatics, paraffins, and olefins over ZSM-48 at 350° C. utilizing 40% ethanol feed.

FIG. 9 is a graph showing total hydrocarbon (HC) yields for aromatics, paraffins, and olefins over ZSM-48 at 450° C. utilizing 40% ethanol feed.

FIG. 10 is a graph showing olefin yields as a percent of the total hydrocarbon product for ZSM-48 at 300° C. utilizing 40% ethanol feed.

FIG. 11 is a graph showing olefin yields as a percent of the total hydrocarbon product for ZSM-48 at 350° C. utilizing 40% ethanol feed.

FIG. 12 is a graph showing olefin yields as a percent of the total hydrocarbon product for ZSM-48 at 450° C. utilizing 40% ethanol feed.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, systems and methods are provided for integrated conversion of biomass to ultimately form naphtha and/or diesel boiling range products. The integrated conversion can include an initial conversion of biomass to alcohols, such as by fermentation, followed by conversion of alcohols to olefins and then olefins to naphtha, jet, and diesel boiling range compounds, with high selectivity for formation of diesel boiling range compounds.

In some aspects, the integrated conversion process can be facilitated by using a common catalyst for both the conversion of alcohols to olefins and the conversion of olefins to naphtha and/or diesel boiling range compounds. For example, ZSM-48 (an MRE zeotype framework structure catalyst) can be used as the catalyst for both conversion of alcohols to olefins and for oligomerization of olefins with increased selectivity for formation of diesel boiling range products.

In some aspects, still further benefits can be achieved by using a common regenerator to regenerate the common catalyst for the conversion of alcohols to olefins and the conversion of olefins to higher boiling products. Thus, a single regenerator can be used to provide regenerated ZSM-48 catalyst (or regenerated catalyst based on another framework type) for both conversion processes.

Additionally or alternately, in some aspects, integrated conversion of biomass to diesel boiling range products can be performed while achieving an unexpectedly high level of energy efficiency for a biomass-to-fuel conversion process. When targeting formation of diesel boiling range products, the carbon efficiency of an integrated conversion process can be reduced relative to the carbon efficiency for a process focused on production of naphtha boiling range products. However, it has unexpectedly been discovered that the energy efficiency of such a process for production of diesel boiling range products can be improved relative to the energy efficiency of a corresponding process for production of naphtha boiling range products.

One of the difficulties with conversion of biomass to naphtha and/or diesel boiling range fuels is that a large number of conversion steps are required. The first conversion step corresponds to a conversion of biomass to alcohols. While alcohols can correspond to relatively high octane components, there are many difficulties with incorporating large quantities of alcohol into a fuel product for general commercial use. As a result, it is often desirable to perform additional conversion steps to create naphtha and/or diesel boiling range products that can be used in conventional systems.

Although additional conversion steps can be performed, each additional conversion step requires a reactor and accompanying support equipment, such as catalyst regenerators. Providing such additional equipment can substantially increase the costs for producing a fuel product from the alcohol. In various aspects, the amount of additional equipment and/or associated costs can be reduced or minimized by using a common catalyst for the additional conversion steps. For production of diesel boiling range fuels, an MRE zeotype framework catalyst such as ZSM-48 can be used. ZSM-48 can provide several advantages in this type of integrated configuration. First, when used for alcohol conversion to olefins, ZSM-48 can produce substantial amounts of C₃₊ olefins. Performing oligomerization on C₂ olefins results in primarily formation of naphtha (or lower) boiling range compounds while C₃₊ olefins can be oligomerized, in the presence of a catalyst such as ZSM-48, to form substantial amounts of diesel boiling range products.

In this discussion, a liquid product is defined as a product that is substantially in the liquid phase at 20° C. and ˜100 kPa-a. Similarly, a gas product is defined as a product that is substantially in the gas phase at 20° C. and ˜100 kPa-a.

In this discussion, the naphtha boiling range is defined as ˜29° C. (roughly boiling point of C₅ compound) to 170° C. A naphtha boiling range fraction is defined as a fraction having a T10 distillation point of 29° C. or more and a T90 distillation point of 170° C. or less. The diesel boiling range is defined as 170° C. to 343° C. A diesel boiling range fraction is defined as a fraction having a T10 distillation point of 170° C. or more and a T90 distillation point of 343° C. or less.

In this discussion, some feeds, fractions, or products may be described based on a fraction that boils below or above a specified distillation point. For example, a 170° C.− product corresponds to a product that substantially contains components with a boiling point (at standard temperature and pressure) of 170° C. or less. Similarly, a 170° C.+ product corresponds to a product that substantially contains components with a boiling point of 170° C. or more. Substantially containing components within a boiling range is defined herein as containing 90 vol % or more of components within the boiling range, optionally 95 vol % or more, such as a product where all components are within the specified boiling range.

As defined herein, the term “hydrocarbonaceous” includes compositions or fractions that contain hydrocarbons and hydrocarbon-like compounds that may contain heteroatoms typically found in petroleum or renewable oil fraction and/or that may be typically introduced during conventional processing of a petroleum fraction. Heteroatoms typically found in petroleum or renewable oil fractions include, but are not limited to, sulfur, nitrogen, phosphorous, and oxygen. Other types of atoms different from carbon and hydrogen that may be present in a hydrocarbonaceous fraction or composition can include alkali metals as well as trace transition metals (such as Ni, V, or Fe).

As used herein, a zeotype refers to a crystalline material having a porous framework structure built from tetrahedral atoms connected by bridging oxygen atoms. Examples of known zeotype/zeolite frameworks are given in the “Atlas of Zeolite Frameworks” published on behalf of the Structure Commission of the International Zeolite Association”, 6th revised edition, Ch. Baerlocher, L. B. McCusker, D. H. Olson, eds., Elsevier, New York (2007) and the corresponding web site, http://www.iza-structure.org/databases/. Under this definition, a zeolite can refer to aluminosilicates having a zeotype framework type as well, while a zeotype more generally refers crystalline structures having a suitable framework structure that may contain oxides of Si, Al, and/or heteroatoms different from Si and Al. Such heteroatoms can include any heteroatom generally known to be suitable for inclusion in a zeotype framework, such as gallium, boron, germanium, phosphorus, zinc, and/or other transition metals that can substitute for silicon and/or aluminum in a zeotype framework.

As used herein the term ‘moving bed’ reactor means a zone or vessel with contacting of solids and gas flows such that the superficial gas velocity (U) is below the velocity required for dilute-phase pneumatic conveying of solid particles in order to maintain a solids bed with void fraction below 95%. As an example, a moving-bed reactor operating in counter-current flow mode may operate under several flow regimes including settling or moving packed-bed regime (U<U_{mf}), bubbling regime (U_{mf}<U<U_{mb}), slugging regime (U_{mb}<U<U_{c}), transition to and turbulent fluidization regime (U_{c}<U<Uₜᵣ), and fast-fluidization regime (U>Uₜᵣ). As used herein ‘U_{mf}’ is the minimum fluidization velocity, ‘U_{mb}’ is the minimum bubbling velocity, ‘U_{c}’ is the onset velocity for the transition to turbulent fluidization, and TV is the transport velocity. These different regimes have been described in, for example, Kunii, D., Levenspiel, O., Chapter 3 of Fluidization Engineering, 2″ Edition, Butterworth-Heinemann, Boston, 1991.

As used herein the term ‘fluidized bed’ reactor means a zone or vessel with contacting of solids and gas flows such that the superficial gas velocity (U) is sufficient to fluidize solid particles (i.e., above the minimum fluidization velocity U_{mf}) and is below the velocity required for dilute-phase pneumatic conveying of solid particles in order to maintain a solids bed with void fraction below 95%. Minimum fluidization velocity is discussed in, for example, the Kunii publication noted above.

As used herein the term ‘riser reactor’ means a zone or vessel (such as a vertical cylindrical pipe) used for net upwards transport of solids in fast-fluidization or pneumatic conveying fluidization regimes. Fast fluidization and pneumatic conveying fluidization regimes are characterized by superficial gas velocities (U) greater than the transport velocity (Uₜᵣ). Fast fluidization and pneumatic conveying fluidization regimes are also described in the Kunii publication noted above.

### Configuration Example

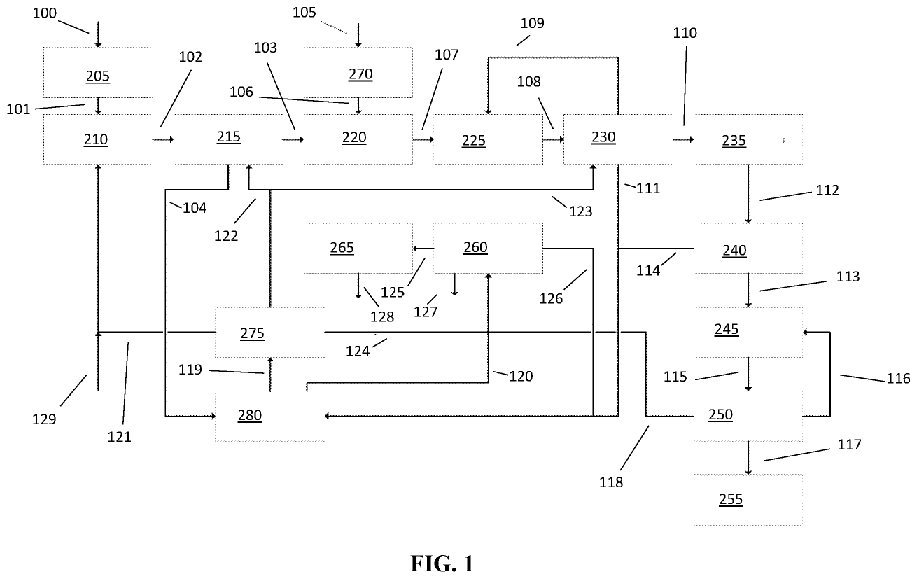

FIG. 1 shows an example of an integrated configuration for converting biomass into naphtha and/or diesel boiling range products. In the example configuration shown in FIG. 1, the raw material biomass 100 arrives at the plant and is milled 205 to an appropriate size. Examples of suitable types of biomass include, but are not limited to, corn stover, switchgrass, or a combination thereof. More generally, any convenient type of cellulosic biomass could be used.

The milled biomass 101 can then be transported to the feed preparation step 210 where depending on the moisture level, the feed can be slurried. The optionally slurry biomass feed 102 is then sent to the deconstruction section 215 to be broken into components, such as hemicellulose, cellulose, and lignin. The deconstruction 215 can be performed by any convenient method, such as by using acid and steam. The water 104 that is removed in this process can be sent to the anaerobic and aerobic digester block 280 for removal of organic impurities. Some of the hemicellulose in the optionally slurried biomass feed 102 can be converted to pentose sugar during the biomass deconstruction.

The effluent stream 103 containing hemicellulose, cellulose, lignin, and some pentose sugar can be further treated in a hydrolysis section 220 to release the remaining monosaccharides. Hydrolysis can be performed, for example, using an enzyme such cellulase. In the example configuration shown in FIG. 1, the enzyme can be produced in enzyme production block 270 on-site and is fed 106 to the hydrolysis section 220. Feeds 105 for enzyme production, such as glucose and nutrients are fed to the enzyme production block 270. Using enzymatic hydrolysis can convert the cellulose to hexose sugars such as glucose and also yield more pentose sugars.

The hydrolyzed feed 107 including the additional hexose and pentose sugars can then be fed to the fermentation reactor 225 for conversion (by fermentation) to ethanol. The resulting lignin-rich ethanol product stream 108 is then sent to a recovery unit 230. In recovery unit 230, insoluble solids 111 such as lignin can first be separated and sent to the digester 280 for conversion into fuel (biogas) for generation of high pressure superheated steam. This results in a solid-free ethanol stream that also includes a substantial content of water. The ethanol concentration in the resulting solid-free ethanol stream can range between 20 wt % to 40 wt % prior to any separation to remove water. In some aspects, the solid-free ethanol stream with the substantial water content can be used as a feed for conversion of ethanol to olefins. In other aspects, such as in the configuration shown in FIG. 1, the solid-free ethanol stream can be sent to a series of separators (such as distillation columns and/or molecular sieve separators) to remove at least a portion of the excess water. This can result in production of concentrated ethanol, excess water, and a vapor effluent that also contains some ethanol. In some aspects, the concentrated ethanol stream can have a concentration ranging from 40 wt % to 80 wt %. In other aspects, such as the aspect shown in FIG. 1, the concentrated ethanol stream can have a water content of 80 wt % or higher. As an example, one option can be to distill the solid free ethanol stream to form a concentrated ethanol stream that approaches near to the azeotrope value of roughly 95 wt % ethanol. This can correspond to a concentration ethanol stream with an ethanol concentration of 80 wt % to 95 wt %, or 85 wt % to 95 wt %, or 80 wt % to 90 wt %. Because the alcohol conversion reaction is relatively tolerant of the presence of excess water, a distillation that increases the concentration of ethanol can improve reaction efficiency without having to take the additional steps required for distilling ethanol past the azeotrope concentration. The removed excess water can be included as part of the insoluble solids stream 111 that is sent for treatment in digester 280. The vapor effluent from the fermentation reactor and distillation columns can contain some ethanol which is recovered in a water wash column and recycled back 109 to the beer well at the outlet of the fermentation reactor.

In the example configuration shown in FIG. 1, a relatively concentrated ethanol (and/or other alcohol) stream 110 with a concentration ranging from 80 wt % to 99+ wt % is fed to an ethanol (and/or other alcohol) conversion block 235 that converts the ethanol to olefins over a zeolite catalyst in a reactor. In some aspects, the concentration of ethanol and/or other alcohol introduced into the alcohol conversion reaction can be 85 wt % or more, or 90 wt % or more, such as up to being substantially free of water (i.e., water content of less than 1.0 wt %, or less than 0.1 wt %). Reducing the water content in the alcohol stream 110 can reduce or minimize steaming of the catalyst used in the alcohol conversion block 235. In various aspects, the catalyst can correspond to ZSM-48 and/or another MRE framework structure catalyst. In some aspects, the operating conditions for alcohol conversion block 235 (such as an ethanol conversion block) can range, for example, from 1.0 to 5.0 bar pressure (˜100 kPa-a to 500 kPa-a), 350° C. to 500° C., and 0.5 hr⁻¹ to 1 hr⁻¹ weight hourly space velocity (WHSV). It is noted that alcohol conversion block 235 can also include an associated catalyst regenerator to allow, for example, for removal of coke formed on the catalyst during alcohol conversion.

The olefin products 112 from the alcohol conversion block 235 can be sent to an olefin recovery block 240. In olefin recovery block 240, one or more separations (such as distillations) can be performed to separate water and methane from an olefin-rich stream 113. It is noted that the alcohol conversion process can generate substantial volumes of water, independent of any water content that was present in the feed to the alcohol conversion block 235. Due in part to the nature of the oligomerization reaction, it can be desirable to reduce the water content of olefin-rich stream 113 to 10 wt % or less, or 5.0 wt % or less, or 3.0 wt % or less, such as down to having substantially no water content (1.0 wt % or less, or 0.1 wt % or less, or possibly still lower). The separated water and/or methane 114 can be sent to digester block 280 to allow any carbon-containing material to be eventually be added to the fuel for the boiler/steam generator 260.

The remaining relatively olefin-rich stream 113 can then be sent to an oligomerization block 245 to convert the olefin to gasoline boiling range products and/or distillate boiling range products over a zeolite catalyst bed in a reactor. In some aspects, the catalyst can correspond to ZSM-48 and/or another MRE framework structure catalyst. It is noted that oligomerization block 245 can also include an associated catalyst regenerator to allow, for example, for removal of coke formed on the catalyst during oligomerization. In some aspects, the catalyst in the alcohol conversion block 235 and oligomerization block 245 can be the same. Optionally, in aspects where the same catalyst is used in alcohol conversion block 235 and oligomerization block 245, a common regenerator can be used for both alcohol conversion block 235 and oligomerization block 245.

The product of the oligomerization 116 can then be fractionated 250 to form a light end gas, gasoline boiling range products, and diesel boiling range products. In the example configuration shown in FIG. 1, the light end gas 118 is sent to the boiler/steam generator 260. In some aspects, a 320° F. (160° C.) portion 116 of the gasoline range products can be recycled back to the oligomerization block 245 as co-feed to increase the distillate yield and quench to limit the exotherm in the reactor. In such aspects, the recycle rate to oligomerization block 245 can correspond to a 1:1 to 2:1 molar ratio of gasoline to fresh olefin feed. In some aspects, the operating conditions of the oligomerization reactor can range from 20-60 bar (˜2000 kPa-a to ˜6000 kPa-a), 160° C. to 280° C., and 0.5 hr⁻¹ to 2 hr⁻¹ WHSV. Optionally, the distillate and gasoline products 117 from the fractionation unit 250 can be sent to a finishing reactor 255 where the olefin products are hydrogenated.

In FIG. 1, waste water streams are generated by various processes, such as waste water streams 104, 111, 114, and 126. These waste water streams can be treated in anaerobic and aerobic digester block 280. Anaerobic and aerobic digester block 280 can serially treat the waste water stream with at least a first anaerobic digester followed by at least a first aerobic digester. More generally, any convenient combination of anaerobic and aerobic digesters can be used. After digestion, a water effluent stream 119 from digester block 280 can be further treated with water filtration systems 275. A biogas stream 120 is also generated in digester block 280. The biogas stream 120 can be used as fuel for boiler/steam generator 260. In some aspects, the treated water 124 from water filtration systems 275 can also be sent to boiler/steam generator 260 to provide water for the steam generation. Additionally or alternately, water from water filtration systems 275 can be used as water 121 for feed preparation 210, water 122 for biomass deconstruction step 215, and/or water 123 for ethanol recovery step 230. In the boiler/steam generator 260, hydrocarbon waste and lignin are burnt to generate the energy to make high pressure superheated steam 127 to be used in the process and to generate electricity 128 in turboexpander 265. The electricity 128 generated by turboexpander 265 can be used in any convenient manner, such as in the biomass conversion plant and/or as energy for export. Thus, the integrated system can potential provide power to external processes in a manner similar to a co-generation plant/combined heat and power plant. Additional fresh water 129 can be added to the plant if the amount of recycled water is not sufficient.

Integration of Catalytic Processing with Fermentation

In various aspects, lignocellulosic material derived from a biomass source can be converted into carbohydrates and/or sugars by enzymatic processes. Fermentation can then be used to convert the sugars and/or other carbohydrates into alcohols for use as an input to an alcohol conversion process.

The processing steps for converting biomass into sugars and/or fermenting sugars to form alcohols can typically require inputs of heat (for distillation), electricity (for general plant operations), and water (for processing raw materials, cleaning and other processes). One of the advantages of integrating the biomass processing steps (enzyme conversion to sugars, fermentation) with the catalytic processes (alcohol conversion, oligomerization) is that synergies can be achieved between the various process steps. In particular, conventionally upgrading alcohols to diesel poses difficulties, as product yields can be reduced relative to upgrading processes that focus on production of naphtha. However, by integrating the biomass processing steps and the catalytic process steps, the reduction in product yield can be offset by additional generation of electrical power.

In order to characterize the benefits of integration, two types of efficiency can be defined. One type of efficiency is a “carbon efficiency”. The carbon efficiency represents how much of the carbon from the initial biomass ends up in the fuel products generated by the final oligomerization process. In this discussion, carbon efficiency is defined according to Equation (1).

$$Carbon Efficieny = \frac{\sum {( Carbon )}_{Products}}{\sum {( Carbon )}_{All feed}} \qquad ( 1 )$$

As shown in Equation (1), the carbon efficiency is defined as the weight of carbon in the naphtha and diesel boiling range products formed by oligomerization divided by the total weight of carbon in all feeds, which includes carbon in initial biomass feed (such as raw material biomass 100 in FIG. 1) and carbon in the feed 105 for enzyme production. It is noted that auxiliary fuels are not needed for the process, as any heat requirements/power requirements for the process are generated within the integrated process flow.

Another type of efficiency is an “energy efficiency”. The energy efficiency represents the energy value of the products from oligomerization plus any electric power generated by the process relative to the energy value of the initial biomass feed. In this discussion, energy efficiency is defined according to Equation (2).

$$Energy Efficieny = \frac{\sum {( LHV )}_{Products} + Electricity export}{\sum {( LHV )}_{All feed}} \qquad ( 2 )$$

As shown in Equation (2), the energy efficiency is defined as the sum of a) the lower heating value of the naphtha and diesel products from the oligomerization reaction, plus b) any electrical power exported from the process, divided by the lower heating value of all feeds (as defined above). It is noted that the electrical power exported from the process corresponds to electrical power exported after accounting for any electrical power consumed in order to operate the various stages in the integrated process.

Conventionally, the carbon efficiency of processes for converting olefins to naphtha boiling range fractions is higher than the carbon efficiency of processes for converting olefins to distillate. However, in spite of this carbon efficiency advantage, an integrated configuration as shown in FIG. 1 can provide a superior energy efficiency. For example, attempting to increase the diesel boiling range products from oligomerization can also result in increased production of light ends. In an integrated configuration, these light ends can be used as fuel, for example, for generating steam for a turboexpander. Such light ends do not contribute to carbon efficiency, but in an integrated system, such light ends can contribute to the energy efficiency.

### Catalyst Regeneration

The conversion of ethanol to olefins and/or the oligomerization of olefins to naphtha boiling range compounds and distillate boiling range compounds can be performed in any convenient type of reactor(s). Suitable reactors may include fixed bed reactors, moving bed reactors, fluidized bed reactors, and riser reactors. Fixed bed reactors can provide advantages for simplifying the operation of the reactor. However, as implied by the name, catalyst cannot be readily removed from a fixed bed reactor during operation. As a result, the catalyst activity and run length for a fixed bed reactor can be dictated at least in part by the nature of any catalyst deactivation that occurs as processing is performed in the fixed bed reactor.

In contrast to a fixed bed reactor, in reactors such as moving bed reactors, fluidized bed reactors, and riser reactors, catalyst can be added to and subtracted from the reactor on a regular or routine basis. When catalyst is removed from a reactor, at least a portion of such removed catalyst can be exposed to regeneration conditions. By controlling the regeneration conditions and controlling the rate of catalyst removal/addition for a reactor, the average level of catalyst deactivation in a reactor can be controlled. This can allow a reactor to be operated at a level of catalyst deactivation that is advantageous for performing the reaction within the reactor.

In some aspects, the conversion of ethanol to olefins can be performed in a reactor that allows for catalyst removal and regeneration during operation (as well as subsequent return of catalyst to the reactor). Examples of such reactors are moving bed reactors, riser reactors, and/or fluidized bed reactors. Additionally or alternately, the oligomerization of olefins to naphtha/diesel boiling range compounds can be performed in a reactor that allows for catalyst removal, regeneration, and return during operation, such as a moving bed reactor, riser reactor, or fluidized bed reactor.

In configurations where catalyst regeneration is performed, one option can be to have a separate regenerator for the ethanol (or other alcohol) conversion stage and the oligomerization stage. This can be beneficial, for example, when different catalysts are used in the stages. In other aspects, the same type of catalyst can be used in the ethanol (or other alcohol) conversion stage and the oligomerization stage. In such aspects, separate regenerators can be used for each stage, or a common regenerator can be used to provide regeneration for catalyst from both the alcohol (such as ethanol) conversion stage and the oligomerization stage.

In aspects where a common regenerator is used, an example of a suitable catalyst is an MRE framework structure catalyst, such as ZSM-48. Using ZSM-48 as both an alcohol conversion catalyst and as an oligomerization catalyst can provide several advantages. First, as an alcohol conversion catalyst, ZSM-48 has relatively high selectivity for formation of C₃₊ olefins. This is in contrast to catalysts such as ZSM-5, which tend to have higher selectivity for formation of C₂ olefins. Such higher carbon number (C₃₊) olefins can be beneficial for increasing the yield of diesel boiling range products when performing oligomerization with some types of oligomerization catalysts. Additionally, during oligomerization, ZSM-48 has relatively high selectivity for converting higher carbon number (C₃₊) olefins into diesel boiling range products. Thus, when attempting to convert ethanol to diesel boiling range products, ZSM-48 can provide benefits both for forming higher carbon number olefins and for converting such olefins to diesel boiling range products, and can therefore be used in both the olefin generation and oligomerization steps, operated under different conditions.

For either separate regeneration zones per reaction stage or a common regeneration zone for both alcohol conversion and oligomerization, a regeneration zone can correspond to one or more reactors (such as a plurality of reactors connected in parallel) operated as a fixed bed, a fluidized bed, an ebullating bed, a settling bed, a riser reactor or a combination thereof. In some aspects, a regeneration zone can be operated at the minimum temperature required to remove the required amount of coke at a target residence time. Preferably, the temperature should not exceed the point at which metal oxide volatilization occurs and/or the conversion catalyst substrate undergoes rapid deterioration. Typically, a regeneration zone temperature can be from 400° C. to 700° C., or 550° C. to 650° C. Catalyst residence time in the regeneration zone can also be reduced or minimized to reduce catalyst aging rate and/or increase or maximize percent of time the catalyst spends in the reactor doing useful work. In embodiments, the average residence time of catalyst particles in the regeneration zone (either in the regenerator and/or in other conduits or vessels different from the ethanol conversion reactor and the oligomerization reactor) may be between 0.1 and 500 minutes, or between 1.0 minutes to 300 minutes, or between 1.0 minutes to 100 minutes, or between 1 and 20 minutes.

### Catalysts

In various embodiments, alcohols and ethers may be converted into olefins in the presence of a conversion catalyst under conversion conditions. Additionally, the resulting olefins can be converted to naphtha boiling range products and diesel boiling range products in the presence of an oligomerization catalyst. One option for the conversion catalyst and the oligomerization catalyst is to use a catalyst based on ZSM-48 (and/or another type of MRE framework catalyst). It is noted that using a common catalyst (such as ZSM-48) for both ethanol conversion and oligomerization can allow the same pool of catalyst to be used for both reaction stages. This can allow for potential synergies such as using a common regenerator for regenerating the catalyst used in both reactors.

More generally a variety of zeolite and/or zeotype catalysts can be suitable as a conversion catalyst and/or an oligomerization catalyst. The conversion catalyst may comprise a zeolite (or other zeotype) in its original crystalline form or after formulation into catalyst extrudates, such as by extrusion. When a formulated catalyst is used, the formulated catalyst can include a binder, or can be formulated without a binder.

For catalysts that include a binder, zeolite crystals can be combined with a binder, such as, for example, one or more of Al₂O₃, TiO₂, ZrO₂, SiO₂, SiO₂/Al₂O₃, and MgO to form bound catalysts. Generally, a binder can be present in an amount between 1 wt % to 90 wt % relative to a weight of the catalyst, or 10 wt % to 90 wt %, or 20 wt % to 90 wt %, or 1 wt % to 70 wt %, or 10 wt % to 70 wt % or 20 wt % to 70 wt %, or 1 wt % to 40 wt %, or 10 wt % to 40 wt %, or 20 wt % to 40 wt %. Combining the zeolite and the binder can generally be achieved, for example, by mulling a mixture of the zeolite and binder (optionally an aqueous mixture) and then extruding the mixture into catalyst pellets.

In some aspects, a binder for formulating a catalyst can be selected so that the resulting bound catalyst has a micropore surface area of at least about 290 m²/g, for example at least about 300 m²/g or at least about 310 m²/g. Optionally but preferably, a suitable binder can be a binder with a surface area of about 200 m²/g or less, for example about 175 m²/g or less or about 150 m²/g or less. Unless otherwise specified, the surface area of the binder is defined herein as the combined micropore surface area and mesopore surface area of the binder.

Alternatively, a zeolite (or more generally zeotype) catalyst can be formulated into catalyst particles without including a binder. A process for producing zeolite extrudates in the absence of a binder is disclosed in, for example, U.S. Pat. No. 4,582,815, the entire contents of which are incorporated herein by reference.

In some aspects, the catalyst can include a zeotype framework (such as a zeolite) having a Constraint index of 1-12 (as defined in U.S. Pat. No. 4,016,218). Some suitable zeotypes can include zeotypes having an MFI or MEL framework, such as ZSM-5 or ZSM-11. ZSM-5 is described in detail in U.S. Pat. No. 3,702,886 and RE29,948. ZSM-11 is described in detail in U.S. Pat. No. 3,709,979. Other zeotypes can include, but are not limited to, the zeotype frameworks corresponding to ZSM-12 (U.S. Pat. No. 3,832,449); ZSM-22 (U.S. Pat. No. 4,556,477); ZSM-23 (U.S. Pat. No. 4,076,842); ZSM-34 (U.S. Pat. No. 4,079,095) ZSM-35 (U.S. Pat. No. 4,016,245); ZSM-48 (U.S. Pat. No. 4,397,827); ZSM-57 (U.S. Pat. No. 4,046,685); and ZSM-58 (U.S. Pat. No. 4,417,780). Non-limiting examples of SAPO and AlPO molecular sieves can include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AlPO-5, AlPO-11, AlPO-18, AlPO-31, AlPO-34, AlPO-36, AlPO-37, and AlPO-46.

Another option for characterizing a zeotype/zeolite or other molecular sieve is based on the nature of the ring channels in the zeotype/zeolite. The ring channels in a zeotype framework structure can be defined based on the number of atoms including in the ring structure that forms the channel. In some embodiments, a zeotype framework structure can include at least one ring channel based on a 10-member ring. In such aspects, the framework structure preferably does not have any ring channels based on a ring larger than a 10-member ring. Examples of suitable framework structures having a 10-member ring channel but not having a larger size ring channel include EUO, FER, IMF, LAU, MEL, MFI, MFS, MTT, MWW, NES, PON, SFG, STF, STI, TON, TUN, MRE, and PON framework types.

In some alternative embodiments, the zeotype/zeolite can be a molecular sieve that includes an 8-member ring channel (small pore molecular sieves), a 10-member ring channel (as described above), or a 12-member ring channel (large pore molecular sieves), but does not have any ring channels based on a ring larger than a 12-member ring. In such aspects, suitable large pore molecular sieves can include those having AFI, AFS, ATO, ATS, *BEA, BEC, BOG, BPH, CAN, CON, EMT, EON, EZT, FAU, GME, GON, IFR, ISV, -*ITN, IWR, IWW, LTL, MAZ, MEI, MOR, MOZ, MSE, MTW, OFF, OKO, OSI, SAF, SAO, SEW, SFE, SFO, SSF, SSY, and USI framework types. In such aspects, suitable small pore molecular sieves can include those having the AEI, AFT, AFX, ATT, DDR, EAB, EPI, ERI, KFI, LEV, LTA, MER, MON, MTF, PAU, PHI, RHO, and SFW framework types.

Generally, a zeolite having the desired activity can have a silicon to aluminum molar ratio of 10 to 300, or 15 to 100, or 20 to 80, or 20 to 40. In some aspects, the silicon to aluminum ratio can be at least 20, or at least 30, or at least 40. In such embodiments, the silicon to aluminum ratio can optionally be 100 or less, or 80 or less, or 60 or less, or 50 or less, or 40 or less. Typically, reducing the silicon to aluminum ratio in a zeolite can result in a zeolite with a higher acidity, and therefore higher activity for cracking of hydrocarbon or hydrocarbonaceous feeds, such as petroleum feeds. With respect to conversion of alcohols and/or ethers to olefins, such increased cracking activity due to a decrease in the silicon to aluminum ratio may result in increased formation of residual carbon or coke during the conversion reaction. Such residual carbon can deposit on the zeolite conversion catalyst, leading to a change in the properties of the catalyst over time. Having a silicon to aluminum ratio of at least 40, or at least 50, or at least 60, can reduce/minimize the amount of additional residual carbon formed due to the acidic or cracking activity of the conversion catalyst.

It is noted that the molar ratio described herein is a ratio of silicon to aluminum. If a corresponding ratio of silica to alumina were described, the corresponding ratio of silica (SiO₂) to alumina (Al₂O₃) would be twice as large, due to the presence of two aluminum atoms in each alumina stoichiometric unit compare to only one silicon atom in the silica stoichiometric unit. Thus, a silicon to aluminum ratio of 10 corresponds to a silica to alumina ratio of 20.

In some optional aspects, the zeolite conversion catalyst employed herein can further be characterized by at least one or at least two of the following properties: (a) a mesoporosity of greater than 20 m²/g, or greater than 30 m²/g, and/or less than 150 m²/g, less than 145 m²/g; (b) a microporous surface area of at least 140 m²/g, or at least 145 m²/g. Additionally, or alternatively, a conversion catalyst may have a combined micropore and mesopore surface area of at least 30 m²/g, or at least 50 m²/g.

Of these properties, mesoporosity can be determined by several factors for a given zeolite, including the crystal size of the zeolite. Microporous surface area is determined by the pore size of the zeolite and the availability of the zeolite pores at the surfaces of the catalyst particles. Producing a zeolite conversion catalyst with the desired minimum mesoporosity and microporous surface area should be well within the expertise of anyone of ordinary skill in zeolite chemistry. It is noted that mesopore or external surface area and micropore surface area can be characterized, for example, using adsorption-desorption isotherm techniques within the expertise of one of skill in the art, such as the BET (Brunauer Emmett Teller) method.

It is noted that the micropore surface area can be characterized for either zeolite crystals or a catalyst formed from the zeolite crystals. In various aspects, the micropore surface area of a self-bound catalyst or a catalyst formulated with a separate binder can be at least about 290 m²/g, for example at least about 300 m²/g, at least about 310 m²/g, at least about 320 m²/g, or at least about 330 m²/g. Typically, a formulation of zeolite crystals into catalyst particles (either self-bound or with a separate binder) can result in some loss of micropore surface area relative to the micropore surface area of the zeolite crystals. Thus, to provide a catalyst having the desired micropore surface area, the zeolite crystals can also have a micropore surface area of at least about 290 m²/g, for example at least about 300 m²/g, or at least about 310 m²/g. As a practical matter, the micropore surface area of a zeolite crystal and/or a corresponding self-bound or bound catalyst as described herein can be less than about 1000 m²/g, and typically less than about 750 m²/g. Additionally or alternately, the micropore surface area of a catalyst (self-bound or with a separate binder) can be about 105% or less of the micropore surface area of the zeolite crystals in the catalyst, and typically about 100% or less of the micropore surface area of the zeolite crystals in the catalyst, for example from about 80% to 100% of the micropore surface area of the zeolite crystals in the catalyst. In some embodiments, the micropore surface area of a catalyst can be at least about 80% of the micropore surface area of the zeolite crystals in the catalyst, for example at least about 85%, at least about 90%, at least about 95%, at least about 97%, or at least about 98%, and/or about 100% or less, for example about 99% or less, about 98% or less, about 97% or less, or about 95% or less.

When used in the present conversion catalyst, the zeolite can be present at least partly in the hydrogen (active) form. Depending on the conditions used to synthesize the zeolite, this may correspond to converting the zeolite from, for example, the sodium form. This can readily be achieved, for example, by ion exchange to convert the zeolite to the ammonium form followed by calcination in air or an inert atmosphere at a temperature of about 400° C. to about 700° C. to convert the ammonium form to the active hydrogen form. Alternatively, methods for directly converting a sodium form zeolite to a hydrogen form zeolite can also be used. Such methods are well known to the person of ordinary skill in the art.

In some aspects, the conversion catalyst can include and/or be enhanced by one or more metals selected from groups 1 to 14 of the periodic table.

The metal can be incorporated into the zeolite by any convenient method known in the art, such as by impregnation or by ion exchange. After impregnation or ion exchange, the metal-enhanced catalyst may be treated in air or an inert atmosphere at a temperature of about 400° C. to about 700° C. The amount of metal can be related to the molar amount of aluminum present in the zeolite. In some embodiments, the molar amount of the metal can correspond to about 0.1 to about 1.3 times the molar amount of aluminum in the zeolite. In some embodiments, the molar amount of metal can be at least about 0.1 times the molar amount of aluminum in the zeolite, for example at least about 0.2 times, at least about 0.3 times, or at least about 0.4 times. Additionally, or alternatively, the molar amount of metal can be about 1.3 times or less relative to the molar amount of aluminum in the zeolite, for example about 1.2 times or less, about 1.0 times or less, or about 0.8 times or less. Still further additionally or alternately, the amount of metal can be expressed as a weight percentage of the conversion catalyst, such as having at least about 0.1 wt. % of metal, at least about 0.25 wt. %, at least about 0.5 wt. %, at least about 0.75 wt. %, or at least about 1.0 wt. %. Additionally, or alternatively, the amount of metal can be about 20 wt. % or less, for example about 10 wt. % or less, about 5 wt. % or less, about 2.0 wt. % or less, about 1.5 wt. % or less, about 1.2 wt. % or less, about 1.1 wt. % or less, or about 1.0 wt. % or less.

In some aspects, the conversion catalyst can include one or more metals from groups 12 to 14 of the periodic table and thus include the metal zinc. In additional or alternate embodiments, the conversion catalyst can include one or more metals from groups 1 and 2 of the periodic table. The total weight of the metals can be 0.1 wt % to 10 wt % based on the total weight of the conversion catalyst, or 0.1 wt % to 5.0 wt %, or 0.1 wt % to 2.0 wt %, or 0.5 wt % to 10 wt %, or 0.5 wt % to 5.0 wt %, or 0.5 wt % to 2.0 wt %, or 1.0 wt % to 10 wt %, or 1.0 wt % to 5.0 wt %.

To form a metal-enhanced conversion catalyst, a self-bound (or bound) catalyst can, for example, be impregnated via incipient wetness with a solution containing the desired metal for impregnation, such as one or more of Zn, Ga, B, Ca, Ti, V, Fe, Cu, Mo, Ru, Pd, Rh, Ir, Nb, W, Re, and Pt. The impregnated catalyst can then be dried overnight at roughly 120° C., followed by calcination in air, such as for roughly 3 hours at 540° C. More generally, a transition metal can be incorporated into the zeolite crystals and/or catalyst at any convenient time, such as before or after ion exchange to form H-zeolite crystals, or before or after formation of an extrudate. In some embodiments that are preferred from a standpoint of facilitating manufacture of a zeolite catalyst, the transition metal can be incorporated into the catalyst (such as by impregnation or ion exchange) after formation of the catalyst by extrusion or another convenient method.

In some optional aspects, a single conversion catalyst (used for both alcohol conversion and oligomerization) or a separate oligomerization catalyst can be used that includes 0.1 wt % to 10 wt % of Ni as a supported metal, or 0.5 wt % to 2.0 wt %. The Ni can be added to the oligomerization catalyst by any convenient method, such as by spray coating, ion exchange, or incipient wetness. In such aspects, the single conversion catalyst or the oligomerization catalyst can preferably include an MRE framework structure (such as ZSM-48).

### Reaction Conditions and Products—Alcohol Conversion and Olefin Oligomerization

Suitable and/or effective conditions for performing a conversion reaction for conversion of alcohols (such as ethanol) to olefins may include average reaction temperatures of 200° C. to 550° C. (or 250° C. to 550° C., or 300° C. to 550° C., or 350° C. to 550° C., or 400° C. to 500° C.), total pressures between 10 psig (^{˜}70 kPa-g) to 400 psig (^{˜}2700 kPa-g), or 50 psig (^{˜}350 kPa-g) to 350 psig (^{˜}2400 kPa-g), or 100 psig 0700 kPa-g) to 300 psig (^{˜}2100 kPa-g), and an alcohol space velocity between 0.1 h⁻¹ to 10 h⁻¹ based on weight of alcohol relative to weight of catalyst. For example, the average reaction temperature may be at least 200° C., or at least 250° C., or at least 300° C., or at least 350° C., or at least 400° C., or at least 450° C. Additionally or alternately, the average reaction temperature can be 550° C. or less, or 500° C. or less, or 450° C. or less, or 400° C. or less. In this specification, average reaction temperature is defined as the average of the temperature at the reactor inlet and the temperature at the reactor outlet for the reactor where the conversion reaction is performed. In some aspects, where lower pressures are used, the pressure can correspond to 70 kPa-g to 700 kPa-g. As another example, the total pressure can be at least 70 kPa-g, or at least 350 kPa-g, or at least 500 kPa-g, or at least 700 kPa-g, or at least 1000 kPa-g. Additionally or alternately, the total pressure can be 3000 kPa-g or less, or 2700 kPa-g or less, or 2400 kPa-g or less, or 2100 kPa-g or less.

Suitable and/or effective conditions for performing an oligomerization reaction can include average reactor temperatures of 125° C. to 250° C. (or 125° C. to 200° C., or 150° C. to 220° C.); total pressures between 100 psig (˜0.7 MPa-g) to 3000 psig (˜20.7 MPa-g), or 100 psig (˜0.7 MPa-g) to 2000 psig (˜13.8 MPa-g), or 200 psig (˜1.3 MPa-g) to 1000 psig (˜6.9 MPa-g), or 70 psig (˜500 kPa-g) to 180 psig (˜1200 kPa-g); and an olefin space velocity between 0.1 h⁻¹ to 5 h⁻¹ based on weight of olefins relative to weight of catalyst. In some aspects, the lower pressure operating range for oligomerization can be beneficial for performing oligomerization on an olefin-containing portion of an oxygenate conversion effluent without having to perform prior compression on the portion of the conversion effluent. One option for selecting suitable oligomerization conditions can be to select conditions effective for conversion of a desired percentage of the ethylene within the feed to oligomerization. For example, the effective conditions for oligomerization can comprise conditions for conversion of 50 wt % or more of the ethylene in the feed to oligomerization, or 70 wt % or more, or 90 wt % or more.

### Example 1: Conversion of Ethanol to Olefins with ZSM-48

Ethanol conversion experiments were performed in a ˜10 cc reactor, with a fixed bed of ˜2 g zeolite catalyst diluted in sand. The ethanol was 100% ethanol. The ethanol feed rate was 5 cc/h for all experiments, and pressure was varied during the runs are described below. The WHSV for all experiments was ˜2.

ZSM-48 catalyst was contacted with ethanol in the fixed bed reactor operating at 350° C. or 450° C. and at a pressure of about 15 psig. The ethanol was fed to the zeolite catalyst bed and the effluent from the reactor periodically analyzed for product composition. Ethanol conversion was ˜100% throughout the runs.

Gas phase analysis was performed with an online GC. The liquid products were separated by density into aqueous and hydrocarbon components. The aqueous phase was analyzed by density measurement and the hydrocarbon phase was analyzed by GC.

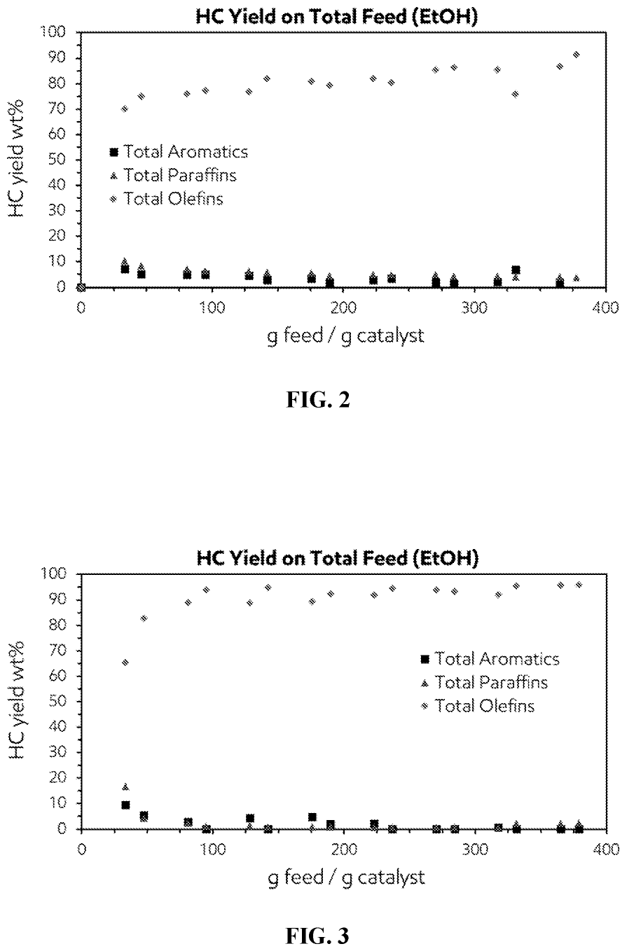

FIG. 2 and FIG. 3 illustrate the weight % hydrocarbon yield against gram ethanol feed per gram catalyst at 350° C. and 450° C. respectively.

Referring to FIG. 2, initially, olefins comprise 70% of the hydrocarbon products, with this rising to 90% at around 375 g feed/g catalyst.

These data highlight the ability of ZSM-48, as a structure with 1D channels and 10 membered rings, to inhibit the cyclization required to form aromatics and remain within the olefin methylation cycle. It is notable that this was possible despite the C₂ starting unit in ethanol, which could potentially ethylate olefins and more quickly produce larger olefins more prone to cyclization. However, the inhibition of the cyclic transition state likely holds and olefins were observed as the primary products.

At the higher 450° C. temperature (FIG. 3), the production of olefins was even more pronounced, with olefin yield rapidly increasing to >90 wt % and remaining there for the duration of the experiment. This is significant, as the higher temperature would perhaps be expected to favor higher aromatics production than at 350° C., as the hydrogen transfer reaction required to dehydrogenate naphthenes to aromatics is more likely at higher temperature. However, the opposite was observed, which is possibly a result of the combination of the inhibition of ring formation in the first place by the small, 1D channels of ZSM-48, coupled with the more active cracking of higher olefins to lighter olefins at the higher temperature.

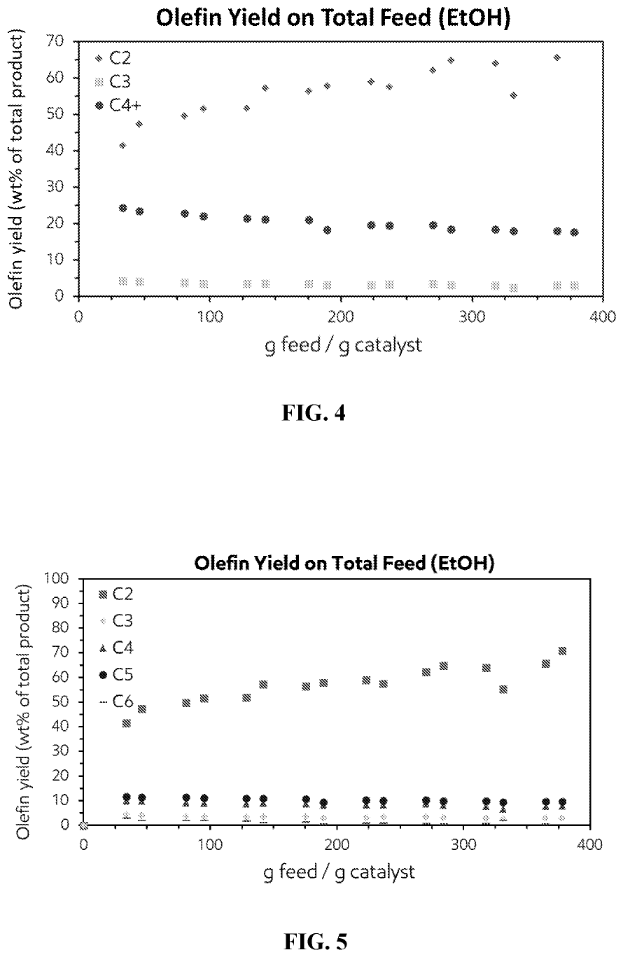

The olefin distribution using ZSM-48 is depicted in FIGS. 4, 5 and 6. Referring first to FIG. 4 (350° C.), ethylene was the primarily olefinic product, accounting for 40-65 wt % of the total hydrocarbon product. C₄₊ olefins made up 20-25 wt % of the hydrocarbon products and propylene was <5 wt %. This distribution is interesting as ethanol dehydration to ethylene appeared to be fast, compared to the methylation/ethylation of ethylene to higher olefins, indicated by the high ethylene production. However, some alkylation of ethylene did occur to produce higher olefins, indicating the possibility that cracking of higher olefins, which was likely fast relative to ring-closing to form naphthenes and aromatics, leading to a substantial portion of the ethylene as well. This is suggested through a further breakdown of the olefin production by carbon number (FIG. 5), which shows higher selectivity to C₅ olefins over C₄ olefins.

The cracking required to produce the C₁ unit necessary for methylation to the odd numbered species indicates this is occurring in the system, which likely contributes to the higher production of C₅ over C₄ olefins. However, this mechanism would be expected to yield C₃ olefins as well, of which there are very few in the products.

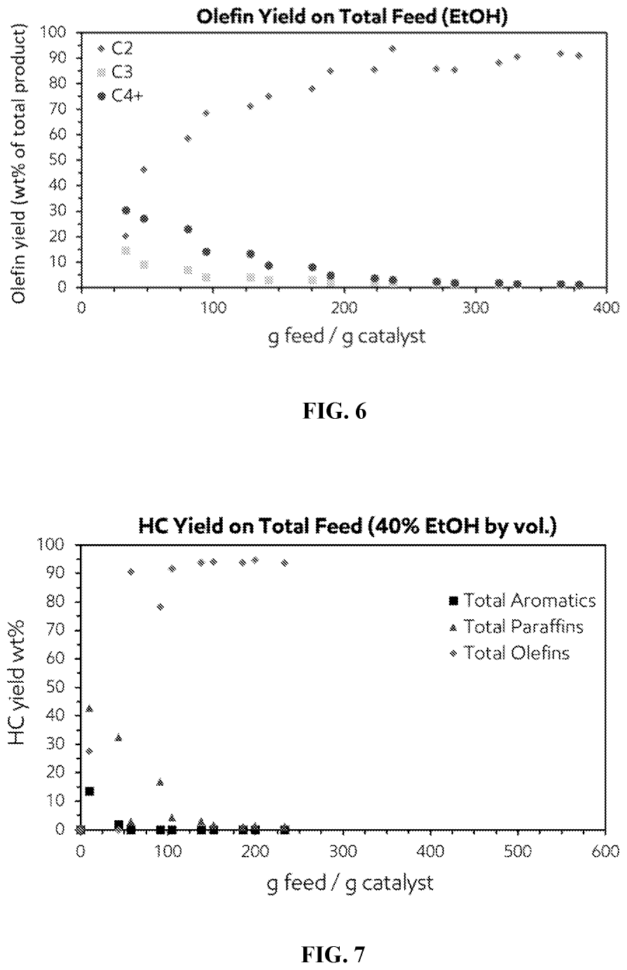

Olefin yields at 450° C. are illustrated in FIG. 6. This was quite different from 350° C. during the initial stages of the reaction and C₄₊ olefins were the primary product at slightly more than 30 wt % of the hydrocarbon products, with an additional 15 wt % propylene. The higher temperature in this reaction likely better enabled the alkylation of olefins, leading to a higher production of C₃₊ olefins.

Interestingly, C₅ olefins are more prevalent in the products than C₄ olefins suggesting that cracking of higher olefins is occurring in order to produce significant quantities of olefins with an odd number of carbons. It has been proposed that the production of propylene stems from cracking of higher olefins, possibly 4-methyl-1-pentene. However, as the reaction progressed and the catalyst began to coke, ethylene again became the dominant product, eventually reaching 85 wt % of the hydrocarbon product stream.

As a point of comparison, an experiment with methanol (450° C., 15 psig, similar ZSM-48 catalyst) produced a maximum of ˜55-60% olefins. Ethanol feed produced significantly higher total olefins products of 60-90 wt %, depending on time on stream. Additionally, the distribution of olefins was different. With methanol, the C₂, C₃, and C₄₊ olefin distribution was (all values are wt % of total product): 4.7, 22.0, 27.8, respectively. This differs from the comparable olefin distribution of ˜42, 4.5, and 25%, respectively, with ethanol, in that the higher olefin production was primarily driven by a large increase in ethylene production that more than offset the decrease in propylene production. This is likely driven by the C₂ unit already present in ethanol. Overall, the total C₄₊ olefins production, which are the most readily oligomerized to distillate, was similar between methanol and ethanol feeds.

Utilizing the enabling technology of, for example, a moving bed reactor, the operating window can essentially be chosen, with the catalyst operating continuously at a specific level of coking. In this case, rapidly reactivating the ZSM-48 catalyst and operating at low coking levels may be optimal, as this allows for the greatest production of C₃₊ olefins useful for oligomerization to fuels.

In Table 1, the “optimal” conditions for producing olefins at each temperature are shown for ZSM-48. Given the potential to select operating conditions and an operating window with, for example, a moving bed reactor, these yields may be achieved in a commercial process. In Table 1, ‘P’=paraffins, ‘A’=aromatics and ‘O’=olefins.

TABLE 1

| | ZSM-48 | |
|---|---|---|
| | % product | |
| Temperature | | C3 + |
| (° C.) | P/A/O | olefins |
| 350 | 10/7/70 | 29 |
| 450 | 17/10/66 | 45 |

### Example 2: Conversion of 40% Ethanol to Olefins with ZSM-48

ZSM-48 catalyst was tested with a feed of 40% ethanol in water. Results were collected at 300, 350, and 450° C. for the 40% ethanol feed.

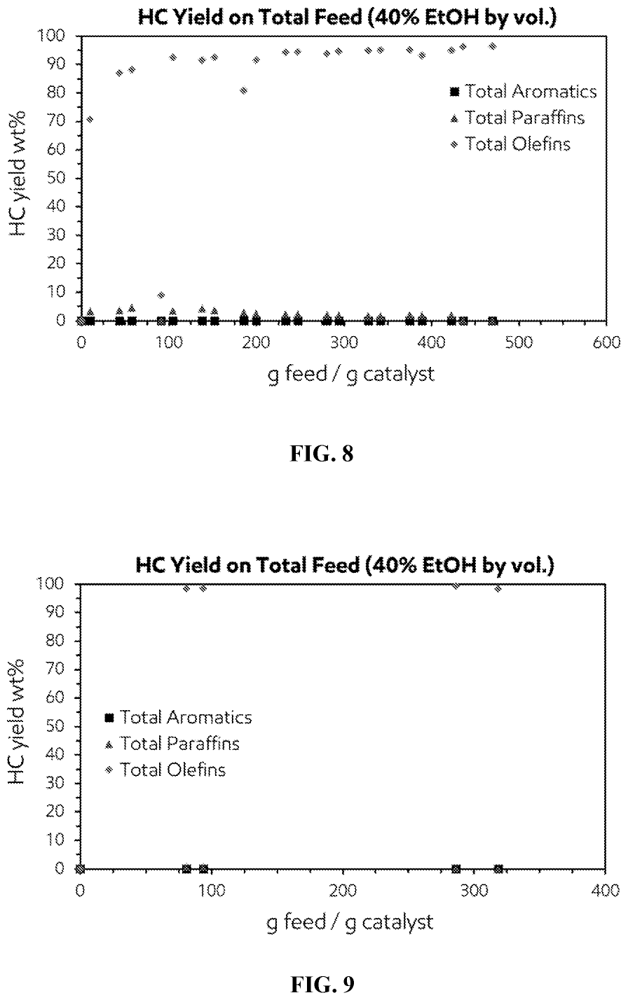

Overall hydrocarbon yields are depicted in FIG. 7, FIG. 8, and FIG. 9 for the 300° C., 350° C., and 450° C. runs, respectively, all of which were conducted at 15 psig (˜100 kPa-g) pressure for the duration of each run.

From FIG. 7, it is evident that ZSM-48 initially showed some conversion of the aqueous ethanol to paraffins and aromatics, but this rapidly decreased to almost exclusively olefins. This was likely the result of modification of the catalyst in the presence of water. Corresponding results for experiments at 350° C. (FIG. 8) and 450° C. (FIG. 9) indicate that this modification was quite rapid, with high quantities of olefins being produced at all points during the runs. The 450° C. data suffered from some experimental issues, but the results can be inferred from the data points obtained, in which ˜100% olefins were produced as hydrocarbon products.

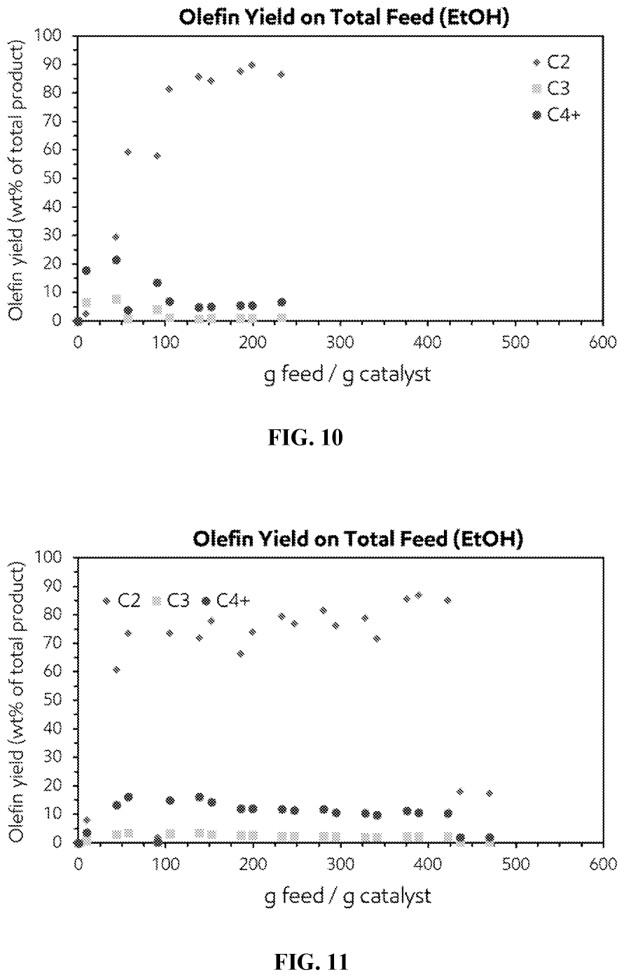

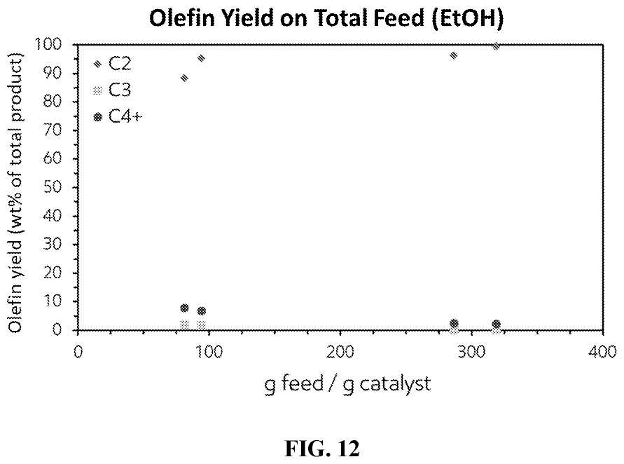

Notably, this conversion to olefins occurred despite the presence of a significant amount of water. The results in FIG. 10, FIG. 11, and FIG. 12 demonstrate that significant quantities of C₃₊ olefins can be produced, especially at lower temperatures.

At 300° C. (FIG. 10), C₃₊ olefin yields were quite high, being >25% of the total product, while ethylene yield was ˜3% at the beginning of the run. After some time, this changed to primarily ethylene production, but C₃₊ olefin production remained high for the first 100 g feed/g catalyst of the run. This demonstrates that it is possible to create a significant advantage in the process conditions and energy input requirements for bioethanol conversion. It is noted that by using a moving bed and/or other type of reaction system where catalyst can be withdrawn, regeneration can be performed on the catalyst during operation. This can allow the average catalyst lifetime in a reactor to be maintained at a desired or target level, such as maintaining an average catalyst lifetime below 100 g feed/g catalyst. Thus, regeneration of the catalyst during a run can allow increased C₃₊ olefin yields to be achieved for longer reactor run times. Such regeneration can also assist with maintaining a stable yield and/or can prevent excessive steam dealumination.

While not quite as high in C₃₊ olefins production at 350° C. (FIG. 11), their production remained steady in the 10-20 wt. % range over the course of the experiment, again indicating the potential long-term operation of a moving bed unit for ethanol conversion to olefins. At 450° C. (FIG. 12), the C₃₊ olefin yields were quite low (<10 wt %), decreasing to very low after some time on stream.

### Example 3—Energy Efficiency for Integrated Production of Diesel Boiling Range Products

A configuration similar to the configuration shown in FIG. 1 was used as the basis for modelling of the carbon efficiency and energy efficiency for integrated conversion of biomass to fuels boiling range products. In one set of calculations, product formation was based on using ZSM-5 as the catalyst for both the alcohol conversion step and the oligomerization step. In another set of calculations, ZSM-48 was used as the catalyst for alcohol conversion and oligomerization.

In the model, public literature values were used to model the processes for conversion of biomass into sugars and subsequent fermentation of the sugars. The fermentation process was modelled based on conversion of sugars into ethanol. The resulting fermentation products were then modelled as being fully separated to form an ethanol feed for conversion that included less than 1.0 wt % water. As shown in FIG. 1, all waste streams including carbon-containing materials were passed into a digester block for eventual incorporation into fuel for the boiler, in order to raise steam for electric power generation and for steam usage in the plant.

The ethanol conversion block and oligomerization block were modelled based on experimental data for conversion and oligomerization over ZSM-5 and ZSM-48 catalysts, respectively. The ethanol conversion processes were modelled at a temperature of 450° C. This resulted in preferential formation of C₂ olefins for ethanol conversion over ZSM-5. At this temperature, the yield (in weight percent) of paraffins/aromatics/olefins for the ZSM-5 ethanol conversion was 39/34/20. The percentage of C₃₊ olefins relative to the total olefin yield was 12 wt %. The product distribution and C₃₊ olefin content for the ZSM-48 ethanol conversion is shown in the 450° C. row of Table 1 above. The oligomerization conditions were selected to maximize the yield of naphtha for the ZSM-5 catalyst and the yield of diesel for the ZSM-48 catalyst, based on the olefin-containing feeds that were provided to the oligomerization block. It is understood that some diesel is also formed during ZSM-5 oligomerization, while some naphtha is formed during ZSM-48 oligomerization.

It is noted that the biomass conversion stage and fermentation stage portions of the model were identical for the calculations using ZSM-5 or ZSM-48 as the catalyst in the catalytic stages. Thus, any differences in carbon efficiency or energy efficiency are due to the differences in the resulting product slates generated from using the different catalysts.

The modelling was performed based on a processing plant size capable of processing 2000 metric tons of dry biomass feed per day. Table 2 shows the resulting yields of naphtha and diesel boiling range products, along with the carbon efficiency and energy efficiency.

TABLE 2
Efficiencies and Yields for Integrated Fuels Production

| | Energy | Carbon | Naphtha, | Diesel, |
|---|---|---|---|---|
| | eff. % | eff. % | kbd | kbd |
| ZSM-48 | 27% | 12% | 0.11 | 0.97 |
| ZSM-5 | 26% | 14% | 0.99 | 0.42 |

As shown in Table 2, production of naphtha using ZSM-5 resulted in a higher carbon efficiency. This is not surprising, as production of diesel boiling range compounds has a higher reliance on the presence of C₃₊ olefins. However, in spite of the lower carbon efficiency, it was unexpectedly found that the energy efficiency of the ZSM-48 based conversion process was actually higher. This is due in part to the ability to produce additional excess electrical power based on the carbon not incorporated into the fuel products. Thus, by using an integrated system, an unexpectedly high energy efficiency can be achieved for conversion of biomass with selectivity for formation of diesel boiling range products.

### Additional Embodiments

Embodiment 1. A method for converting biomass, comprising: exposing a feed comprising biomass to an enzymatic conversion process to form an effluent comprising one or more sugars; exposing at least a portion of the one or more sugars to fermentation conditions to form a fermented effluent comprising one or more alcohols; exposing a conversion stage feed comprising at least a portion of the one or more alcohols to a catalyst comprising ZSM-48, an MRE framework structure, or a combination thereof, under alcohol conversion conditions to form a conversion effluent comprising C₃₊ olefins, the alcohol conversion stage comprising a moving bed reactor, a fluidized bed reactor, a riser reactor, or a combination thereof; exposing at least a portion of the conversion effluent to oligomerization conditions in the presence of additional catalyst comprising ZSM-48, an MRE framework structure, or a combination thereof, to form at least a diesel boiling range fraction, the oligomerization stage comprising a moving bed reactor, a fluidized bed reactor, a riser reactor, or a combination thereof; regenerating at least a portion of the catalyst comprising ZSM-48, an MRE framework structure, or a combination thereof, and at least a portion of the additional catalyst comprising ZSM-48, an MRE framework structure, or a combination thereof, in a regenerator vessel to form regenerated catalyst; and passing at least a first portion of the regenerated catalyst into the alcohol conversion stage and at least a second portion of the regenerated catalyst into the oligomerization stage.

Embodiment 2. The method of Embodiment 1, wherein the conversion effluent further comprises a methane-containing fraction and wherein the fermentation conditions further form a fermentation residue, the method further comprising: forming a fuel from at least a portion of the methane-containing fraction from the conversion effluent and at least a portion of the fermentation residue; and combusting at least a portion of the fuel to generate steam, to power a turbine for generating electricity, or a combination thereof.

Embodiment 3. A method for converting biomass, comprising: exposing a feed comprising biomass to an enzymatic conversion process to form an effluent comprising one or more sugars; exposing at least a portion of the one or more sugars to fermentation conditions to form a fermented effluent comprising one or more alcohols, and a fermentation residue; exposing a conversion stage feed comprising at least a portion of the one or more alcohols to a catalyst comprising ZSM-48, an MRE framework structure, or a combination thereof, under alcohol conversion conditions in an alcohol conversion stage to form a conversion effluent; separating an olefin-containing fraction comprising C₃₊ olefins and a methane-containing fraction from the conversion effluent; exposing at least a portion of the conversion effluent to oligomerization conditions in the presence of a catalyst comprising ZSM-48, an MRE framework structure, or a combination thereof, in an oligomerization stage to form at least a diesel boiling range fraction; forming a fuel from at least a portion of the methane-containing fraction from the conversion effluent and at least a portion of the fermentation residue; and combusting at least a portion of the fuel to generate steam, to power a turbine for generating electricity, or a combination thereof.

Embodiment 4. The method of Embodiment 3, wherein forming a fuel from the at least a portion of the fermentation residue comprises passing the at least a portion of the fermentation residue into a digester to form a digester biogas, and incorporating at least a portion of the digester biogas into the fuel.

Embodiment 5. The method of Embodiment 3 or 4, further comprising regenerating catalyst from the alcohol conversion stage and catalyst from the oligomerization stage in a common regenerator, or wherein the alcohol conversion stage comprises a first regenerator and the oligomerization stage comprises a second regenerator.

Embodiment 6. The method of any of the above embodiments, wherein the feed comprising biomass comprises lignin, cellulose, hemicellulose, or a combination thereof, or wherein the method further comprising forming the feed comprising biomass by exposing a raw biomass feed to deconstruction conditions in the presence of acid and steam, or a combination thereof.

Embodiment 7. The method of any of the above embodiments, wherein the conversion stage feed comprises 80 wt % or more of the at least a portion of the one or more alcohols, relative to a weight of the conversion stage feed.

Embodiment 8. The method of any of the above embodiments, wherein the conversion stage feed comprises 40 wt % to 80 wt % of the at least a portion of the one or more alcohols, relative to a weight of the conversion stage feed.

Embodiment 9. The method of any of the above embodiments, wherein the one or more alcohols comprise ethanol.

Embodiment 10. The method of any of the above embodiments, wherein exposing the at least a portion of the conversion effluent to the oligomerization conditions further forms a naphtha boiling range product, and wherein a weight of the diesel boiling range product is greater than a weight of the naphtha boiling range product.

Embodiment 11. The method of Embodiment 10, wherein at least a portion of the naphtha boiling range product is combusted to generate steam, to power a turbine for generating electricity, or a combination thereof.

Embodiment 12. The method of any of the above embodiments, wherein the at least a portion of the conversion effluent comprises 10 wt % or less of water.

Embodiment 13. The method of any of the above embodiments, wherein the at least a portion of the conversion effluent comprises 20 wt % or more of C₃₊ olefins relative to a weight of olefins in the at least a portion of the conversion effluent.

Embodiment 14. A system for converting biomass, comprising: a deconstruction stage for conversion of raw biomass into biomass comprising lignin, cellulose, hemicellulose, or a combination thereof, the deconstruction stage comprising a deconstructed biomass outlet; an enzymatic conversion stage comprising an enzyme inlet, an enzymatic conversion effluent outlet, and a biomass inlet in fluid communication with the deconstructed biomass outlet; a fermentation stage comprising a fermentation inlet in fluid communication with the enzymatic conversion outlet, a recovered vapor inlet, and a fermented effluent outlet; a recovery stage comprising a recovery inlet in fluid communication with the fermented effluent outlet, a solids outlet, an alcohol outlet, and a vapor outlet, the vapor outlet being in fluid communication with the recovered vapor inlet; an alcohol conversion stage comprising an alcohol conversion inlet in fluid communication with the alcohol outlet, an alcohol conversion effluent outlet, a conversion catalyst inlet, and a conversion catalyst outlet, the alcohol conversion stage comprising a moving bed reactor, a fluidized bed reactor, a riser reactor, or a combination thereof, the alcohol conversion stage comprising a catalyst comprising ZSM-48, an MRE framework structure, or a combination thereof; an alcohol separation stage comprising an alcohol separation inlet in fluid communication with the alcohol conversion effluent outlet, a separated water outlet, and an olefin-containing effluent outlet; an oligomerization stage comprising an oligomerization inlet in fluid communication with the olefin-containing effluent outlet, a recycled product inlet, an oligomerized product outlet, an oligomerization catalyst inlet, and an oligomerization catalyst outlet, the oligomerization stage comprising a moving bed reactor, a fluidized bed reactor, a riser reactor, or a combination thereof, the oligomerization stage comprising additional catalyst comprising ZSM-48, an MRE framework structure, or a combination thereof; an oligomerized product separation stage comprising a product separation inlet in fluid communication with the oligomerized product outlet, a product outlet, and a recycled product outlet in fluid communication with the recycled product inlet; a regeneration stage comprising a first regenerator inlet in solids flow communication with the conversion catalyst outlet, a second regenerator inlet in solids flow communication with the oligomerization catalyst outlet, a first regenerator outlet in solids flow communication with the conversion catalyst inlet, and a second regenerator outlet in solids flow communication with the oligomerization catalyst inlet.

Embodiment 15. The system of Embodiment 14, wherein the enzymatic conversion stage comprising an enzymatic conversion process to form an effluent comprising one or more sugars, and wherein the fermentation stage comprising fermentation conditions to form a fermented effluent comprising one or more alcohols.

Additional Embodiment A. Performing the method of any of Embodiments 1 to 13 in a system according to Embodiment 14 or 15.

Additional Embodiment B. An oligomerized product formed according to the method of any of Embodiments 1 to 13 or using the system of any of Embodiments 14 or 15.

When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

The present invention has been described above with reference to numerous embodiments and specific examples. Many variations will suggest themselves to those skilled in this art in light of the above detailed description. All such obvious variations are within the full intended scope of the appended claims.

What is claimed is:

1. A method for converting biomass, comprising:
exposing a feed comprising biomass to an enzymatic conversion process to form an effluent comprising one or more sugars;
exposing at least a portion of the one or more sugars to fermentation conditions to form a fermented effluent comprising one or more alcohols;
exposing a conversion stage feed comprising at least a portion of the one or more alcohols to a catalyst comprising ZSM-48, an MRE framework structure, or a combination thereof, under alcohol conversion conditions to form a conversion effluent comprising C₃₊ olefins, the alcohol conversion stage comprising a moving bed reactor, a fluidized bed reactor, a riser reactor, or a combination thereof;
exposing at least a portion of the conversion effluent to oligomerization conditions in the presence of additional catalyst comprising ZSM-48, an MRE framework structure, or a combination thereof, to form at least a diesel boiling range fraction, the oligomerization stage comprising a moving bed reactor, a fluidized bed reactor, a riser reactor, or a combination thereof;
regenerating at least a portion of the catalyst comprising ZSM-48, an MRE framework structure, or a combination thereof, and at least a portion of the additional catalyst comprising ZSM-48, an MRE framework structure, or a combination thereof, in a regenerator vessel to form regenerated catalyst; and
passing at least a first portion of the regenerated catalyst into the alcohol conversion stage and at least a second portion of the regenerated catalyst into the oligomerization stage.

2. The method of claim 1, wherein the conversion effluent further comprises a methane-containing fraction and wherein the fermentation conditions further form a fermentation residue, the method further comprising:
forming a fuel from at least a portion of the methane-containing fraction from the conversion effluent and at least a portion of the fermentation residue; and
combusting at least a portion of the fuel to generate steam, to power a turbine for generating electricity, or a combination thereof.

3. The method of claim 1, wherein the feed comprising biomass comprises lignin, cellulose, hemicellulose, or a combination thereof, or wherein the method further comprising forming the feed comprising biomass by exposing a raw biomass feed to deconstruction conditions in the presence of acid and steam, or a combination thereof.

4. The method of claim 1, wherein the conversion stage feed comprises 80 wt % or more of the at least a portion of the one or more alcohols, relative to a weight of the conversion stage feed.

5. The method of claim 1, wherein the conversion stage feed comprises 40 wt % to 80 wt % of the at least a portion of the one or more alcohols, relative to a weight of the conversion stage feed.

6. The method of claim 1, wherein the one or more alcohols comprise ethanol.

7. The method of claim 1, wherein exposing the at least a portion of the conversion effluent to the oligomerization conditions further forms a naphtha boiling range product, and wherein a weight of the diesel boiling range product is greater than a weight of the naphtha boiling range product.

8. The method of claim 1, wherein the at least a portion of the conversion effluent comprises 10 wt % or less of water.

9. The method of claim 1, wherein the at least a portion of the conversion effluent comprises 20 wt % or more of C₃₊ olefins relative to a weight of olefins in the at least a portion of the conversion effluent.

10. A method for converting biomass, comprising:
exposing a feed comprising biomass to an enzymatic conversion process to form an effluent comprising one or more sugars;
exposing at least a portion of the one or more sugars to fermentation conditions to form a fermented effluent comprising one or more alcohols, and a fermentation residue;
exposing a conversion stage feed comprising at least a portion of the one or more alcohols to a catalyst comprising ZSM-48, an MRE framework structure, or a combination thereof under alcohol conversion conditions in an alcohol conversion stage to form a conversion effluent;
separating an olefin-containing fraction comprising C₃₊ olefins and a methane-containing fraction from the conversion effluent;
exposing at least a portion of the conversion effluent to oligomerization conditions in the presence of a catalyst comprising ZSM-48, an MRE framework structure, or a combination thereof, in an oligomerization stage to form at least a diesel boiling range fraction;
forming a fuel from at least a portion of the methane-containing fraction from the conversion effluent and at least a portion of the fermentation residue; and
combusting at least a portion of the fuel to generate steam, to power a turbine for generating electricity, or a combination thereof.

11. The method of claim 10, wherein forming a fuel from the at least a portion of the fermentation residue comprises passing the at least a portion of the fermentation residue into a digester to form a digester biogas, and incorporating at least a portion of the digester biogas into the fuel.

12. The method of claim 10, further comprising regenerating catalyst from the alcohol conversion stage and catalyst from the oligomerization stage in a common regenerator.

13. The method of claim 10, wherein the alcohol conversion stage comprises a first regenerator, and wherein the oligomerization stage comprises a second regenerator.

14. The method of claim 10, wherein the feed comprising biomass comprises lignin, cellulose, hemicellulose, or a combination thereof, or wherein the method further comprising forming the feed comprising biomass by exposing a raw biomass feed to deconstruction conditions in the presence of acid and steam, or a combination thereof.

15. The method of claim 10, wherein the conversion stage feed comprises 80 wt % or more of the at least a portion of the one or more alcohols, relative to a weight of the conversion stage feed, or wherein the conversion stage feed comprises 40 wt % to 80 wt % of the at least a portion of the one or more alcohols, relative to a weight of the conversion stage feed.

16. The method of claim 10, wherein exposing the olefin-containing feed to the oligomerization conditions further forms a naphtha boiling range product, and wherein a weight of the diesel boiling range product is greater than a weight of the naphtha boiling range product.

17. The method of claim 16, wherein at least a portion of the naphtha boiling range product is combusted to generate steam, to power a turbine for generating electricity, or a combination thereof.

18. The method of claim 10, wherein the at least a portion of the conversion effluent comprises 20 wt % or more of C₃₊ olefins relative to a weight of olefins in the at least a portion of the conversion effluent.

19. A system for converting biomass, comprising:
a deconstruction stage for conversion of raw biomass into biomass comprising lignin, cellulose, hemicellulose, or a combination thereof, the deconstruction stage comprising a deconstructed biomass outlet;
an enzymatic conversion stage comprising an enzyme inlet, an enzymatic conversion effluent outlet, and a biomass inlet in fluid communication with the deconstructed biomass outlet;
a fermentation stage comprising a fermentation inlet in fluid communication with the enzymatic conversion outlet, a recovered vapor inlet, and a fermented effluent outlet;
a recovery stage comprising a recovery inlet in fluid communication with the fermented effluent outlet, a solids outlet, an alcohol outlet, and a vapor outlet, the vapor outlet being in fluid communication with the recovered vapor inlet;
an alcohol conversion stage comprising an alcohol conversion inlet in fluid communication with the alcohol outlet, an alcohol conversion effluent outlet, a conversion catalyst inlet, and a conversion catalyst outlet, the alcohol conversion stage comprising a moving bed reactor, a fluidized bed reactor, a riser reactor, or a combination thereof, the alcohol conversion stage comprising a catalyst comprising ZSM-48, an MRE framework structure, or a combination thereof;
an alcohol separation stage comprising an alcohol separation inlet in fluid communication with the alcohol conversion effluent outlet, a separated water outlet, and an olefin-containing effluent outlet;
an oligomerization stage comprising an oligomerization inlet in fluid communication with the olefin-containing effluent outlet, a recycled product inlet, an oligomerized product outlet, an oligomerization catalyst inlet, and an oligomerization catalyst outlet, the oligomerization stage comprising a moving bed reactor, a fluidized bed reactor, a riser reactor, or a combination thereof, the oligomerization stage comprising additional catalyst comprising ZSM-48, an MRE framework structure, or a combination thereof;
an oligomerized product separation stage comprising a product separation inlet in fluid communication with the oligomerized product outlet, a product outlet, and a recycled product outlet in fluid communication with the recycled product inlet; and
a regeneration stage comprising a first regenerator inlet in solids flow communication with the conversion catalyst outlet, a second regenerator inlet in solids flow communication with the oligomerization catalyst outlet, a first regenerator outlet in solids flow communication with the conversion catalyst inlet, and a second regenerator outlet in solids flow communication with the oligomerization catalyst inlet.

20. The system of claim 19, wherein the enzymatic conversion stage comprising an enzymatic conversion process to form an effluent comprising one or more sugars, and wherein the fermentation stage comprising fermentation conditions to form a fermented effluent comprising one or more alcohols.
